(19) United States
(12) Patent Application Publication
(10) Pub. No.: US 2024/0408585 A1
(43) Pub. Date: Dec. 12, 2024

(54) EPOXIDATION CATALYST

(71) Applicant: BASF SE, Ludwigshafen am Rhein (DE)

(72) Inventors: Andrey KARPOV, Ludwigshafen am Rhein (DE); Christian ALMER, Ludwigshafen am Rhein (DE); Christian WALSDORFF, Ludwigshafen am Rhein (DE); Andreas LEHR, Ludwigshafen am Rhein (DE); Michael NIKOLAY, Ludwigshafen am Rhein (DE); Christian BARTOSCH, Ludwigshafen am Rhein (DE); Mauricio GROBYS, Shanghai (CN)

(21) Appl. No.: 18/274,042

(22) PCT Filed: Jan. 25, 2022
(86) PCT No.: PCT/EP2022/051551

§ 371 Date: Jul. 25, 2023

(30) Foreign Application Priority Data
Jan. 26, 2021 (EP) 21153364.1

(51) Int. Cl. B01J 27/055; B01J 21/04; B01J 23/68; B01J 35/61; B01J 35/63; B01J 37/02; B01J 37/08; C07D 301/10

(52) U.S. Cl. CPC B01J 27/055; B01J 21/04; B01J 23/688; B01J 35/612; B01J 35/633; B01J 35/635; B01J 37/0205; B01J 37/08; C07D 301/10

(57) ABSTRACT

An epoxidation catalyst comprising silver, cesium, rhenium and tungsten deposited on an alumina support, wherein the catalyst comprises 20 to 50 wt.-% of silver, relative to the weight of the catalyst, an amount of cesium C_{cs} of at least 7.5 mmol per kg of catalyst, and an amount of rhenium CR6 and an amount of tungsten Cw so as to meet the following requirements: C_{Re}≥6.7 mmol per kg of catalyst; and C_{Re}+(2χc_{w})≥13.2 mmol per kg of catalyst. The epoxidation catalyst allows for a more efficient conversion of ethylene oxide by gas-phase oxidation of ethylene, particularly displaying high selectivity and high activity. The invention also relates to a process for preparing an epoxidation catalyst as defined in above, comprising i) impregnating an alumina support with a silver impregnation solution; and ii) subjecting the impregnated refractory support to a calcination process; wherein steps i) and ii) are optionally repeated, and at least one silver impregnation solution comprises rhenium, tungsten and cesium. The invention moreover relates to a process for producing ethylene oxide by gas-phase oxidation of ethylene, comprising reacting ethylene and oxygen in the presence of an epoxidation catalyst according to any one of the preceding claims.

The present invention relates to a catalyst effective in the oxidative conversion of ethylene to ethylene oxide, a process for preparing the catalyst, and a process for preparing ethylene oxide by gas-phase oxidation of ethylene by means of oxygen in the presence of the catalyst.

Ethylene oxide is produced in large volumes and is primarily used as an intermediate in the production of several industrial chemicals. For the industrial oxidation of ethylene to ethylene oxide, heterogeneous catalysts comprising metallic silver are used. Catalyst performance may be characterized, e.g., by selectivity, activity and longevity of catalyst activity. Selectivity is the molar fraction of the converted olefin yielding the desired olefin oxide. Even small improvements in selectivity and the maintenance of selectivity over longer time yield huge dividends in terms of process efficiency.

Suitable epoxidation catalysts are generally obtained by depositing metallic silver on a support. Highly selective silver-based epoxidation catalysts have been developed, which extend the selectivity to a value that is closer to the stoichiometric limit. Such highly selective catalysts comprise, in addition to silver as the active component, promoting species for improving the catalytic properties of the catalyst, as described in, e.g., WO 2007/122090 A2 and WO 2010/123856 A1. Examples of promoting species include alkali metal compounds and/or alkaline earth metal compounds, as well as transition metals such as rhenium, tungsten or molybdenum.

WO 2019/154832 A1 describes a catalyst effective in the oxidative conversion of ethylene to ethylene oxide, comprising an alumina support and silver applied to the support, wherein the catalyst comprises defined amounts of cesium, rhenium, tungsten and a specific silicon to earth metal molar ratio.

There remains a need for an epoxidation catalyst which allows for a more efficient conversion of ethylene oxide by gas-phase oxidation of ethylene, particularly a catalyst displaying high selectivity and high activity.

The invention relates to an epoxidation catalyst comprising silver, cesium, rhenium and tungsten deposited on an alumina support, wherein the catalyst comprises 20 to 50 wt.-% of silver, relative to the weight of the catalyst, an amount of cesium cc of at least 7.5 mmol per kg of catalyst, and an amount of rhenium C_{Re} (in mmol/kg) and an amount of tungsten c_{W} (in mmol/kg) so as to meet the following requirements:

$${c}_{Re} \geq 6.7 mmol per kg of catalyst ; and \\ {c}_{Re} + ( 2 \times {c}_{W} ) \geq 13.2 mmol per kg of catalyst .$$

It was found that the optimum amounts of the promoting species to be used are to some extent interdependent. The synergistic effect observed by increasing both cesium c_{Cs} levels and combined rhenium c_{Re} and tungsten c_{W} levels allows for a catalyst with an especially high selectivity and activity. Without wishing to be bound by theory, it is believed that rhenium and tungsten are to some extent interchangeable since both elements primarily exist in a form coordinated four-fold by oxygen, i.e. as perrhenate and tungstate.

The catalyst comprises 20 to 50 wt.-% of silver, relative to the weight of the catalyst. Preferably, the catalyst comprises 25 to 40 wt.-% of silver, relative to the weight of the catalyst. Most preferably, the catalyst comprises 26 to 35% of silver, relative to the weight of the catalyst. A silver content in this range allows for a favorable balance between turnover induced by the catalyst and cost-efficiency of producing the catalyst.

The catalyst comprises an amount of cesium c_{Cs} of at least 7.5 mmol per kg of catalyst. It is particularly preferred that the catalyst comprises an amount of cesium c_{Cs} of 7.5 to 12.4 mmol per kg of catalyst, especially 7.9 to 10.0 mmol per kg of catalyst. Inferior results may be achieved if c_{Cs} exceeds optimum levels.

The catalyst meets the requirement c_{Re}+(2×c_{W})≥13.2 mmol per kg of catalyst. Preferably, the catalyst meets the requirement c_{Re}+(2×c_{W})≥13.4 mmol per kg of catalyst. Most preferably, the catalyst meets the requirement c_{Re}+(2×c_{W})≥13.5 mmol per kg of catalyst or c_{Re}+(2×c_{W})≥14.0 mmol per kg of catalyst.

In particular, the catalyst may meet the requirement c_{Re}+(2×c_{W})=13.2 to 20.8 mmol per kg of catalyst. Preferably, the catalyst meets the requirement c_{Re}+(2×c_{W})=13.4 to 18.8 mmol per kg of catalyst. Most preferably, the catalyst meets the requirement c_{Re}+(2×c_{W})=13.5 to 16.2 mmol per kg of catalyst. Inferior results may be achieved if c_{Re} and/or c_{W} exceed optimum levels.

Preferably, the amount of cesium c_{Cs}, the amount of rhenium c_{Re} and the amount of tungsten c_{W} are selected so that the ratio of c_{Cs} to [c_{Re}+(2×c_{W})] is in the range of 0.4 to 1.0, more preferably 0.5 to 0.7.

The catalyst comprises an amount of rhenium c_{Re} of at least 6.7 mmol per kg of catalyst. Preferably, the catalyst comprises an amount of rhenium c_{Re} of 6.7 to 10.0 mmol per kg of catalyst, especially 6.8 to 8.0 mmol per kg of catalyst.

Preferably, the catalyst comprises an amount of tungsten c_{W} of at least 3.2 mmol per kg of catalyst. It is particularly preferred that the catalyst comprises an amount of tungsten c_{W} of 3.2 to 5.4 mmol per kg of catalyst, especially 3.4 to 4.1 mmol per kg of catalyst.

It was found that the use of a combination of rhenium and tungsten promoter is surprisingly more effective than the use of these promoters alone. In a particularly preferred embodiment, the catalyst comprises an amount of rhenium c_{Re} of 6.7 to 10.0 mmol per kg of catalyst and an amount of tungsten c_{W} of 3.2 to 5.4 mmol per kg of catalyst. It is especially preferred that the catalyst comprises an amount of rhenium c_{Re} of 6.8 to 8.0 mmol per kg of catalyst and an amount of tungsten c_{W} of 3.4 to 4.1 mmol per kg of catalyst.

In some embodiments, the catalyst may include a promoting amount of an alkali metal besides cesium (an “additional alkali metal”) or a mixture of two or more of such alkali metals, such as lithium, sodium, potassium, rubidium, or combinations thereof. The total amount of additional alkali metal, e.g. lithium and/or potassium, will typically range from 10 to 200 mmol/kg, more typically 20 to 150 mmol/kg, most typically 40 to 120 mmol/kg, relative to the total weight of the catalyst. The amount of additional alkali metal is determined by the amount of additional alkali metal contributed by the support and the amount of additional alkali metal contributed by the impregnation solution described below.

Preferably, the catalyst contains at least two light alkali metals, selected from sodium, potassium and lithium. More preferably, the catalyst contains sodium, potassium and lithium.

Preferably, the catalyst comprises an amount of lithium c_{Li} of at least 14.0 mmol per kg of catalyst, preferably 40 to 100 mmol per kg of catalyst.

Preferably, the catalyst comprises an amount of potassium c_{K} of 12.0 mmol or less per kg of catalyst, preferably 3.8 to 8.0 mmol per kg of catalyst.

Preferably, the catalyst comprises an amount of sodium c_{Na} of less than 10 mmol per kg of catalyst, preferably 0.43 to 4.3 mmol per kg of catalyst.

The catalyst may also include a Group IIA alkaline earth metal or a mixture of two or more Group IIA alkaline earth metals. Suitable alkaline earth metal promoters include, for example, beryllium, magnesium, calcium, strontium, and barium or combinations thereof. The amounts of alkaline earth metal promoters can be used in amounts similar to those used for the additional alkali metal promoters.

The catalyst may also include a promoting amount of a main group element or a mixture of two or more main group elements. Suitable main group elements include any of the elements in Groups IIIA (boron group) to VIIA (halogen group) of the Periodic Table of the Elements. For example, the catalyst can include a promoting amount of sulfur, phosphorus, boron, halogen (e.g., fluorine), gallium, or a combination thereof.

In a preferred embodiment, the catalyst comprises sulfur. Preferably, the catalyst comprises an amount of sulfur c_{S} of 10.0 mmol or less per kg of catalyst, preferably 0.1 to 5.0 mmol per kg of catalyst.

The catalyst may also include a promoting amount of a rare earth metal or a mixture of two or more rare earth metals. The rare earth metals include any of the elements having an atomic number of 57 to 103. Some examples of these elements include lanthanum (La), cerium (Ce), and samarium (Sm). The amount of rare earth metal promoters can be used in amounts similar to those used for the transition metal promoters.

The catalyst comprises an alumina support, on which silver, cesium, rhenium and tungsten are deposited. The alumina support typically comprises a high proportion of alumina, i.e. Al₂O₃, and in particular alpha-alumina, for example at least 50 wt.-%, at least 70 wt.-%, at least 80 wt.-%, or at least 90 wt.-%, preferably at least 95 wt.-%, most preferably at least 97.5 wt.-% or at least 99 wt.-%, based on the total weight of the support. Besides alumina, the support may comprise other components, for example binders such as silicates, or other refractory oxides such as zirconia or titania.

The support preferably comprises individual shaped bodies. The size and shape of the individual shaped bodies and thus of the catalyst is selected to allow a suitable packing of the shaped bodies in a reactor tube. The shaped bodies suitable for the catalysts of the invention are preferably used in reactor tubes with a length from 6 to 14 m and an inner diameter from 20 mm to 50 mm. In general, the support is comprised of individual bodies having a maximum extension in the range of 3 to 20 mm, such as 4 to 15 mm, in particular 5 to 12 mm. The maximum extension is understood to mean the longest straight line between two points on the outer circumference of the support.

The shape of the support is not especially limited, and may be in any technically feasible form, depending, e.g., on the extrusion process. For example, the support may be a solid extrudate or a hollow extrudate, such as a hollow cylinder. In another embodiment, the support may be characterized by a multilobe structure. A multilobe structure is meant to denote a cylinder structure which has a plurality of void spaces, e.g., grooves or furrows, running in the cylinder periphery along the cylinder height. Generally, the void spaces are arranged essentially equidistantly around the circumference of the cylinder. Preferably, the support is in the shape of a solid extrudate, such as pellets or cylinders, or a hollow extrudate, such as a hollow cylinder. Alternatively, the support may be shaped by tableting.

The support is typically a porous support and preferably has a water absorption in the range of 0.35 to 0.70 mL/g (mL of water/gram of support). Preferably, the water absorption of the support is in the range of 0.38 to 0.65 mL/g, most preferably 0.41 to 0.60 mL/g. Water absorption refers to vacuum cold water uptake measured at a vacuum of 80 mbar absolute.

Vacuum cold water uptake is determined by placing about 100 g of support (“initial support weight”) in a rotating flask, covering the support with deionized water, and rotating the rotary evaporator for 5 min at about 30 rpm. Subsequently, a vacuum of 80 mbar is applied for 3 min, the water and the support are transferred into a glass funnel, and the support is kept in the funnel for about 5 min with occasional shaking in order to ensure that adhering water runs down the funnel. The support is weighed (“final support weight”). The water absorption is calculated by subtracting the initial support weight from the final support weight and then dividing this difference by the initial support weight. It is believed that a water absorption in the above ranges allows for a favorable duration of exposure of the obtained ethylene oxide to the catalyst.

The support generally has a total Hg pore volume in the range of 0.4 to 3.0 mL/g, preferably 0.45 to 1.0 mL/g, or 0.5 to 0.7 mL/g, as determined by mercury porosimetry. Mercury porosimetry may be performed using a Micrometrics AutoPore IV 9500 mercury porosimeter (140 degrees contact angle, 485 dynes/cm Hg surface tension, 60000 psia max head pressure). The Hg porosity is determined according to DIN 66133 herein, unless stated otherwise. It is believed that a Hg pore volume in this range allows for a favorable duration of exposure of the obtained ethylene oxide to the catalyst.

The support generally has a BET surface area of 1.5 to 10 m²/g, preferably 1.8 to 5 m²/g, or 2.0 to 3 m²/g. The BET method is a standard, well-known method and widely used method in surface science for the measurements of surface areas of solids by physical adsorption of gas molecules. The BET surface is determined according to DIN ISO 9277 herein, unless stated otherwise.

The support may comprise impurities, such as sodium, potassium, iron, silica, magnesium, calcium, zirconium in an amount of 20 to 200 mmol/kg, based on the total weight of the support.

The support preferably does not have wash-coat particles or a wash-coat layer on its surface, so as to fully maintain the porosity and BET surface area of the uncoated support.

The catalyst generally has a total Hg pore volume in the range of 0.15 to 1.0 mL/g, preferably 0.2 to 0.6 mL/g, or 0.3 to 0.5 mL/g, as determined by mercury porosimetry.

Preferably, the catalyst has a BET surface area in the range of 1.6 to 5.0 m²/g, preferably 1.8 to 3.0 m²/g, or 2.0 to 2.8 m²/g.

The invention moreover relates to a process for preparing an epoxidation catalyst as described above, comprising
i) impregnating an alumina support as described above with a silver impregnation solution; and
ii) subjecting the impregnated support to a heat treatment;

wherein steps i) and ii) are optionally repeated, and at least one silver impregnation solution comprises rhenium, tungsten and cesium.

It is understood that all embodiments of the catalyst also apply to the process for preparing the catalyst, where applicable.

In order to obtain a shaped catalyst body having high silver contents, steps i) and ii) can be repeated several times. In that case it is understood that the intermediate product obtained after the first (or subsequent up to the last but one) impregnation/heat treatment cycle comprises a part of the total amount of target Ag and/or promoter concentrations. The intermediate product is then again impregnated with the silver impregnation solution and calcined to yield the target Ag and/or promoter concentrations. It is also possible to establish the desired composition of the catalyst by applying only one impregnation.

Any silver impregnation solution suitable for impregnating a refractory support known in the art can be used. Silver impregnation solutions typically contain a silver carboxylate, such as silver oxalate, or a combination of a silver carboxylate and oxalic acid, in the presence of an aminic complexing agent like a C₁-C₁₀-alkylenediamine, in particular ethylenediamine. Suitable impregnation solutions are described in EP 0 716 884 A2, EP 1 115 486 A1, EP 1 613 428 A1, U.S. Pat. No. 4,731,350 A, WO 2004/094055 A2, WO 2009/029419 A1, WO 2015/095508 A1, U.S. Pat. Nos. 4,356,312 A, 5,187,140 A, 4,908,343 A, 5,504,053 A, WO 2014/105770 A1 and WO 2019/154863. Cesium may suitably be provided as cesium hydroxide. Rhenium and tungsten may suitably be provided as an oxyanion, for example, as a perrhenate or tungstate in salt or acid form.

At least one silver impregnation solution comprises rhenium, tungsten and cesium. It is especially preferred that at least the silver impregnation solution employed in the final impregnation step comprises rhenium, tungsten and cesium.

During heat treatment, liquid components of the silver impregnation solution evaporate, causing a silver compound comprising silver ions to precipitate from the solution and be deposited onto the porous support. At least part of the deposited silver ions is subsequently converted to metallic silver upon further heating. Preferably, at least 70 mol-% of the silver compounds, preferably at least 90 mol-%, more preferably at least 95 mol-% and most preferably at least 99.5 mol-% or at least 99.9 mol-%, i.e. essentially all of the silver ions, based on the total molar amount of silver in the impregnated porous alpha-alumina support, respectively. The amount of the silver ions converted to metallic silver can for example be determined via X-ray diffraction (XRD) patterns.

The heat treatment may also be referred to as a calcination process. Any calcination processes known in the art for this purpose can be used. Suitable examples of calcination processes are described in U.S. Pat. Nos. 5,504,052 A, 5,646,087 A, 7,553,795 A, 8,378,129 A, 8,546,297 A, US 2014/0187417 A1, EP 1 893 331 A1 or WO 2012/140614 A1. Heat treatment can be carried out in a pass-through mode or with at least partial recycling of the calcination gas.

Heat treatment is usually carried out in a furnace. The type of furnace is not especially limited. For example, stationary circulating air furnaces, revolving cylindrical furnaces or conveyor furnaces may be used. In one embodiment, heat treatment constitutes directing a heated gas stream over the impregnated bodies. The duration of the heat treatment is generally in the range of 5 min to 20 h, preferably 5 min to 30 min.

The temperature of the heat treatment is generally in the range of 200 to 800° C., preferably 210 to 650° C., more preferably 220 to 500° C., most preferably 220 to 350° C. Preferably, the heating rate in the temperature range of 40 to 200° C. is at least 20 K/min, more preferably at least 25 K/min, such as at least 30 K/min. A high heating rate may be achieved by directing a heated gas over the impregnated refractory support or the impregnated intermediate catalyst at a high gas flow.

A suitable flow rate for the gas may be in the range of, e.g., 1 to 1,000 Nm³/h, 10 to 1,000 Nm³/h, 15 to 500 Nm³/h or 20 to 300 Nm³/h per kg of impregnated bodies. In a continuous process, the term “kg of impregnated bodies” is understood to mean the amount of impregnated bodies (in kg/h) multiplied by the time (in hours) that the gas stream is directed over the impregnated bodies. It has been found that when the gas stream is directed over higher amounts of impregnated bodies, e.g., 15 to 150 kg of impregnated bodies, the flow rate may be chosen in the lower part of the above-described ranges, while achieving the desired effect.

Determining the temperature of the heated impregnated bodies directly may pose practical difficulties. Hence, when a heated gas is directed over the impregnated bodies during heat treatment, the temperature of the heated impregnated bodies is considered to be the temperature of the gas immediately after the gas has passed over the impregnated bodies. In a practical embodiment, the impregnated bodies are placed on a suitable surface, such as a wire mesh or perforated calcination belt, and the temperature of the gas is measured by one or more thermocouples positioned adjacent to the opposite side of the impregnated bodies which first comes into contact with the gas. The thermocouples are suitably placed close to the impregnated bodies, e.g., at a distance of 1 to 30 mm, such as 1 to 3 mm or 15 to 20 mm from the impregnated bodies.

The use of a plurality of thermocouples can improve the accuracy of the temperature measurement. Where several thermocouples are used, these may be evenly spaced across the area on which the impregnated bodies rest on the wire mesh, or the breadth of the perforated calcination belt. The average value is considered to be the temperature of the gas immediately after the gas has passed over the impregnated bodies. To heat the impregnated bodies to the temperatures as described above, the gas typically has a temperature of 220 to 800° C., more preferably 230 to 550° C., most preferably 240 to 350° C.

Preferably, heating takes place in a step-wise manner. In step-wise heating, the impregnated bodies are placed on a moving belt that moves through a furnace with multiple heating zones, e.g., 2 to 8 or 2 to 5 heating zones. Heat treatment is preferably performed in an inert atmosphere, such as nitrogen, helium, or mixtures thereof, in particular in nitrogen.

Further provided is a process for producing ethylene oxide by gas-phase oxidation of ethylene, comprising reacting ethylene and oxygen in the presence of an epoxidation catalyst as described above.

The epoxidation can be carried out by all processes known to those skilled in the art. It is possible to use all reactors which can be used in the ethylene oxide production processes of the prior art; for example externally cooled shell-and-tube reactors (cf. Ullmann's Encyclopedia of Industrial Chemistry, 5th edition, vol. A-10, pp. 117-135, 123-125, VCH-Verlagsgesellschaft, Weinheim 1987) or reactors having a loose catalyst bed and cooling tubes, for example the reactors described in DE 34 14 717 A1, EP 0 082 609 A1 and EP 0 339 748 A2.

The epoxidation is preferably carried out in at least one tube reactor, preferably in a shell-and-tube reactor. On a commercial scale, ethylene epoxidation is preferably carried out in a multi-tube reactor that contains several thousand tubes. The catalyst is filled into the tubes, which are placed in a shell that is filled with a coolant. In commercial applications, the internal tube diameter is typically in the range of 20 to 40 mm (see, e.g., U.S. Pat. No. 4,921,681 A) or more than 40 mm (see, e.g., WO 2006/102189 A1).

To prepare ethylene oxide from ethylene and oxygen, it is possible to carry out the reaction under conventional reaction conditions as described, e.g., in DE 25 21 906 A, EP 0 014 457 A2, DE 23 00 512 A1, EP 0 172 565 A2, DE 24 54 972 A1, EP 0 357 293 A1, EP 0 266 015 A1, EP 0 085 237 A1, EP 0 082 609 A1 and EP 0 339 748 A2. Inert gases such as nitrogen or gases which are inert under the reaction conditions, e.g. steam, methane, and also optionally reaction moderators, for example halogenated hydrocarbons such as ethyl chloride, vinyl chloride or 1,2-dichloroethane can additionally be mixed into the reaction gas comprising ethylene and molecular oxygen.

The oxygen content of the reaction gas is advantageously in a range in which no explosive gas mixtures are present. A suitable composition of the reaction gas for preparing ethylene oxide can, for example, comprise an amount of ethylene in the range from 10 to 80% by volume, preferably from 20 to 60% by volume, more preferably from 25 to 50% by volume and particularly preferably in the range from 25 to 40% by volume, based on the total volume of the reaction gas. The oxygen content of the reaction gas is advantageously in the range of not more than 10% by volume, preferably not more than 9% by volume, more preferably not more than 8% by volume and very particularly preferably not more than 7.5% by volume, based on the total volume of the reaction gas.

The reaction gas preferably comprises a chlorine-comprising reaction moderator such as ethyl chloride, vinyl chloride or 1,2-dichloroethane in an amount of from 0 to 15 ppm by weight, preferably in an amount of from 0.1 to 8 ppm by weight, based on the total weight of the reaction gas. The remainder of the reaction gas generally comprises hydrocarbons such as methane and also inert gases such as nitrogen. In addition, other materials such as steam, carbon dioxide or noble gases can also be comprised in the reaction gas.

The concentration of carbon dioxide in the feed (i.e. the gas mixture fed to the reactor) typically depends on the catalyst selectivity and the efficiency of the carbon dioxide removal equipment. Carbon dioxide concentration in the feed is preferably at most 3 vol.-%, more preferably less than 2 vol.-%, most preferably less than 1 vol.-%, relative to the total volume of the feed. An example of carbon dioxide removal equipment is provided in U.S. Pat. No. 6,452,027 B1.

The above-described constituents of the reaction mixture may optionally each have small amounts of impurities. Ethylene can, for example, be used in any degree of purity suitable for the gas-phase oxidation according to the invention. Suitable degrees of purity include, but are not limited to, “polymer-grade” ethylene, which typically has a purity of at least 99%, and “chemical-grade” ethylene which typically has a purity of less than 95%. The impurities typically comprise, in particular, ethane, propane and/or propene.

The reaction or oxidation of ethylene to ethylene oxide is usually carried out at elevated catalyst temperatures. Preference is given to catalyst temperatures in the range of 150 to 350° C., more preferably 180 to 300° C., particularly preferably 190 to 280° C. and especially preferably 200 to 280° C. The present invention therefore also provides a process as described above in which the oxidation is carried out at a catalyst temperature in the range 180 to 300° C., preferably 200 to 280° C. Catalyst temperature can be determined by thermocouples located inside the catalyst bed. As used herein, the catalyst temperature or the temperature of the catalyst bed is deemed to be the weight average temperature of the catalyst particles.

The reaction according to the invention (oxidation) is preferably carried out at pressures in the range of 5 to 30 bar. All pressures herein are absolute pressures, unless noted otherwise. The oxidation is more preferably carried out at a pressure in the range of 5 to 25 bar, such as 10 bar to 24 bar and in particular 14 bar to 23 bar. The present invention therefore also provides a process as described above in which the oxidation is carried out at a pressure in the range of 14 bar to 23 bar.

The physical characteristics of the shaped catalyst body, especially the BET surface area and the pore size distribution, may have a significant positive impact on the catalyst selectivity. This effect is especially pronounced when the catalyst is operated at very high work rates, i.e., high levels of olefin oxide production.

The process according to the invention is preferably carried out under conditions conducive to obtain a reaction mixture containing at least 2.3 vol.-% of ethylene oxide. In other words, the ethylene oxide outlet concentration (ethylene oxide concentration at the reactor outlet) is preferably at least 2.3 vol.-%. The ethylene oxide outlet concentration is more preferably in the range of 2.5 to 4.0 vol.-%, most preferably in the range of 2.7 to 3.5 vol.-%.

The oxidation is preferably carried out in a continuous process. If the reaction is carried out continuously, the GHSV (gas hourly space velocity) is, depending on the type of reactor chosen, for example on the size/cross-sectional area of the reactor, the shape and size of the catalyst, preferably in the range from 800 to 10,000/h, preferably in the range from 2,000 to 8,000/h, more preferably in the range from 2,500 to 6,000/h, most preferably in the range from 4,500 to 5,500/h, where the values indicated are based on the volume of the catalyst.

According to a further embodiment, the present invention is also directed to a process for preparing ethylene oxide (EO) by gas-phase oxidation of ethylene by means of oxygen as disclosed above, wherein the EO-space-time-yield measured is greater than 180 kg_{EO}/(m³_{cal}h), preferably to an EO-space-time-yield of greater than 200 kg_{EO}/(m³_{cal}h), such as greater than 250 kg_{EO}/(m³_{cal}h), greater than 280 kg_{EO}/(m³_{cal}h), or greater than 300 kg_{EO}/(m³_{cal}h). Preferably the EO-space-time-yield measured is less than 500 kg_{EO}/(m³_{cal}h), more preferably the EO-space-time-yield is less than 350 kg_{EO}/(m³_{cal}h).

The preparation of ethylene oxide from ethylene and oxygen can advantageously be carried out in a recycle process. After each pass, the newly formed ethylene oxide and the by-products formed in the reaction are removed from the product gas stream. The remaining gas stream is supplemented with the required amounts of ethylene, oxygen and reaction moderators and reintroduced into the reactor. The separation of the ethylene oxide from the product gas stream and its work-up can be carried out by customary methods of the prior art (cf. Ullmann's Encyclopedia of Industrial Chemistry, 5th edition, vol. A-10, pp. 117-135, 123-125, VCH-Verlagsgesellschaft, Weinheim 1987).

The invention will be described in more detail by the subsequent examples.

### Method 1: Analysis of the Total Amount of Ca-, Mg-, Si-, Fe-, K-, and Na-Contents in Alpha-Alumina Supports

### 1A. Sample Preparation for Measurement of Ca, Mg, Si and Fe

About 100 to 200 mg (at an error margin of ±0.1 mg) of a carrier sample were weighed into a platinum crucible. 1.0 g of lithium metaborate (LiBO₂) was added. The mixture was melted in an automated fusion apparatus with a temperature ramp up to a maximum of 1150° C.

After cooling down, the melt was dissolved in deionized water under careful heating. Subsequently, 10 mL of semi-concentrated hydrochloric acid (concentrated HCl diluted with deionized water, volume ratio 1:1, corresponds to about 6 M) were added. Finally, the solution was filled up to a volume of 100 mL with deionized water.

### 1B. Measurement of Ca, Mg, Si and Fe

The amounts of Ca, Mg, Si and Fe were determined from the solution described under item 1A by Inductively Coupled Plasma-Optical Emission Spectroscopy (ICP-OES) using an ICP-OES Varian Vista Pro.

### Parameters:

Wavelengths [nm]: Ca 317.933
   Mg 285.213
   Si 251.611
   Fe 238.204
Integration time: 10 s
Nebulizer: Conikal 3 ml
Nebulizer pressure: 270 kPa
Pump rate: 30 rpm
Calibration: external (matrix-matched standards)

### 1C. Sample Preparation for Measurement of K and Na

About 100 to 200 mg (at an error margin of ±0.1 mg) of a carrier sample were weighed into a platinum dish. 10 mL of a mixture of aqueous concentrated H₂SO₄ (95 to 98%) and deionized water (volume ratio 1:4), and 10 mL of aqueous hydrofluoric acid (40%) were added. The platinum dish was placed on a sand bath and boiled down to dryness. After cooling down the platinum dish, the residue was dissolved in deionized water by careful heating. Subsequently, 5 mL of semi-concentrated hydrochloric acid (concentrated HCl diluted with deionized water, volume ratio 1:1, corresponds to about 6 M) were added. Finally, the solution was filled up to a volume of 50 mL with deionized water.

### 1D. Measurement of K and Na

The amounts of K and Na were determined from the solution described under item 1C by Flame Atomic Absorption Spectroscopy (F-AAS) using an F-AAS Shimadzu AA-7000.

### Parameters:

Wavelengths [nm]: K 766.5 Na 589.0
Gas: Air/acetylene
Slit width: 0.7 nm (K)/0.2 nm (Na)
Nebulizer pressure: 270 kPa
Calibration: external (matrix-matched standards)

### Method 2: Mercury Porosimetry

Mercury porosimetry was performed using a Micrometrics AutoPore IV 9500 mercury porosimeter (140 degrees contact angle, 485 dynes/cm Hg surface tension, 60,000 psia max head pressure). Mercury porosity was determined in accordance with DIN 66133.

### Method 3: BET Surface Area

The BET surface area was determined in accordance with DIN ISO 9277.

### Method 4: Water Absorption

Water absorption refers to vacuum cold water uptake. Vacuum cold water uptake is determined by placing about 100 g of support (“initial support weight”) in a rotating flask, covering the support with deionized water, and rotating the rotary evaporator for 5 min at about 30 rpm. Subsequently, a vacuum of 80 mbar is applied for 3 min, the water and the support are transferred into a glass funnel, and the support is kept in the funnel for about 5 min with occasional shaking in order to ensure that adhering water runs down the funnel.

The support is weighed (“final support weight”). The water absorption is calculated by subtracting the initial support weight from the final support weight and then dividing this difference by the initial support weight.

### Method 5: Side Crush Strength

The side crush strength was determined using an apparatus of the “Z 2.5/T 919” type supplied by Zwick Röll (Ulm), stamp size: 12.7 mm×12.7 mm. Based on measurements of 25 randomly selected shaped bodies, average values were calculated. The measurements of tetralobes were performed along two directions—along the side and along the diagonal. In the measurement along the diagonal, the force is exerted along an axis running through a first outer passageway, the central passageway and a second outer passageway opposite to the first outer passageway. In the measurement along the side, the force is exerted along two axes each running through two outer passageways.

### Supports

### Support A

Support A was an alumina support (>99 wt.-% alpha-alumina) and comprised Si, Ca, Mg, Na, K and Fe as chemical impurities. Support A was obtained from EXACER s.r.l. (Via Puglia 2/4, 41049 Sassuolo (MO), Italy), under the lot number COM 32/19.

Support A comprised silicon in an amount of 14.24 mmol per kg, calcium in an amount of 7.49 mmol per kg, magnesium in an amount of 4.11 mmol per kg, sodium in an amount of 3.04 mmol per kg, potassium in an amount of 5.11 mmol per kg, and iron in an amount of 1.79 mmol per kg, relative to the total weight of the support.

Support A had a total pore volume of 0.52 mL/g and a bimodal pore size distribution with the first log differential pore volume distribution peak at 0.5 μm and the second log differential pore volume distribution peak at 26 μm measured by mercury porosimetry. Furthermore, support A had a BET surface area of 2.2 m²/g. The support had a tetralobe shape with five passageways and displayed a side crushing strength of 96 N.

### Support B

Support B was an alumina support (>99 wt.-% alpha-alumina) and comprised Si, Ca, Mg, Na, K and Fe as chemical impurities. Support B was obtained from EXACER s.r.l. (Via Puglia 2/4, 41049 Sassuolo (MO), Italy), under the lot number COM 55/19.

Support B comprised silicon in an amount of 14.24 mmol per kg, calcium in an amount of 4.99 mmol per kg, magnesium in an amount of 4.11 mmol per kg, sodium in an amount of 4.35 mmol per kg, potassium in an amount of 4.60 mmol per kg, and iron in an amount of 1.79 mmol per kg, relative to the total weight of the support.

Support B had a total pore volume of 0.52 mL/g and a bimodal pore size distribution with the first log differential pore volume distribution peak at 0.5 μm and the second log differential pore volume distribution peak at 26 μm measured by mercury porosimetry. Furthermore, support B had a BET surface area of 2.1 m²/g. The support had a tetralobe shape with five passageways and displayed a side crushing strength of 88 N.

### EXAMPLES

### Example 1—Preparation of Shaped Catalyst Bodies

Shaped catalyst bodies according to Table 1 below were prepared by impregnating support A with a silver impregnation solution.

### 1.1 Production of the Silver Complex Solution

Silver complex solution was prepared according to Production Example 1 of WO 2019/154863 A1. The silver complex solution had a density of 1.529 g/mL, a silver content of 29.3 wt-% and a potassium content of 90 ppmw.

### 1.2. Preparation of Ag-Containing Intermediates

315.3 g of support A were placed into a 2 L glass flask. The flask was attached to a rotary evaporator, which was set under a vacuum pressure of 80 mbar. The rotary evaporator system was set in rotation of 30 rpm. 236.2 g of silver complex solution prepared according to step 1.1 were added onto support A over 15 min under a vacuum pressure of 80 mbar. After addition of the silver complex solution, the rotary evaporator system was continued to rotate under vacuum for a further 15 min. The impregnated support was then left in the apparatus at room temperature (approximately 25° C.) and atmospheric pressure for 1 h and mixed gently every 15 min.

The impregnated material was placed on a net forming 1 to 2 layers (about 100 to 200 g per calcination run). The net was subjected to 23 Nm³/h of air flow, wherein the gas flow was pre-heated to a temperature of 305° C. The impregnated materials were heated up to a temperature of 290° C. at a heating rate of about 30 K/min and then maintained at 290° C. for 8 min to yield Ag-containing intermediate product IA according to Table 2. The temperature was measured by placing three thermocouples at 1 mm below the calcination net. Subsequently, the catalysts were cooled to ambient temperature by removing the intermediate catalyst bodies from the net using an industrial vacuum cleaner.

Ag-containing intermediate product IB was prepared in the same manner, except that support B was used instead of support A. The composition of intermediate product IB is provided in Table 2.

### 1.3. Preparation of Final Catalysts

An amount of Ag-containing intermediate product listed in Table 3 was placed into a 2 L glass flask. The flask was attached to a rotary evaporator, which was set under vacuum pressure of 80 mbar. The rotary evaporator system was set in rotation of 30 rpm. An amount of the silver complex solution listed in Table 3 prepared according to step 1.1 was mixed with amounts of promoter solution I, promoter solution II and promoter solution III as listed in Table 3.

Promoter solution I was obtained by dissolving lithium nitrate (FMC, 99.3%) and ammonium sulfate (Merck, 99.4%) in DI water to achieve target Li and S contents listed in Table 3.

Promoter solution II for catalysts 1-1 to 1-4, 1-7, 1-8, 1-10 and 1-11 was obtained by dissolving tungstic acid (HC Starck, 99.99%) in DI water and cesium hydroxide in water (HC Starck, 50.42%) to achieve target Cs and W contents listed in Table 3.

Promoter solution II for catalysts 1-5 and 1-6 was obtained by dissolving tungstic acid (HC Starck, 99.99%) in a mixture of cesium hydroxide in water (HC Starck, 50.42%) and an aqueous solution of ammonia (2.9 wt.-%) to achieve target Cs and W contents listed in Table 3.

Promoter solution II for catalyst 1-9 was obtained by dissolving tungstic acid (HC Starck, 99.99%) in a mixture of cesium hydroxide in water (HC Starck, 50.42%) and an aqueous solution of ammonia (4.6 wt.-%) to achieve target Cs and W contents listed in Table 3.

Promoter solution III for catalysts 1-1 and 1-2 was obtained by dissolving ammonium perrhenate (Buss & Buss Spezialmetalle GmbH, 99.9%) in deionized water (“DI water”) to achieve a target Re content of 3.7 wt.-%.

Promoter solution III for catalysts 1-3 to 1-11 was obtained by dissolving ammonium perrhenate (Buss & Buss Spezialmetalle GmbH, 99.9%) in a 29 wt.-% aqueous solution of ethylene diamine to achieve a target Re content of 10.0 wt.-%.

The combined impregnation solution containing silver complex solution, promoter solutions I, II, and III and an amount of DI water as listed in Table 3 was stirred for 5 min. The combined impregnation solution was added onto an amount of the silver-containing intermediate product prepared according to step 1.2 listed in Table 3 over 15 min under a vacuum pressure of 80 mbar. After addition of the combined impregnation solution, the rotary evaporator system was continued to rotate under vacuum for another 15 min. The impregnated support was then left in the apparatus at room temperature (about 25° C.) and atmospheric pressure for 1 h and gently mixed every 15 min.

The impregnated material was placed on a net forming 1 to 2 layers (about 100 to 250 g per calcination run). The net was subjected to 23 Nm³/h nitrogen flow (oxygen content: <20 ppm), wherein the gas flows were pre-heated to a temperature of 305° C. The impregnated materials were heated up to a temperature of 290° C. at a heating rate of about 30 K/min and then maintained at 290° C. for 7 min to yield catalysts according to Table 1. The temperatures were measured by placing three thermocouples at 1 mm below the calcination net. Subsequently, the catalysts were cooled to ambient temperature by removing the catalyst bodies from the net using an industrial vacuum cleaner.

TABLE 1
Catalyst composition (Ag-contents are reported in percent by weight of total
catalyst, dopant values are reported in mmol/kg of total catalyst).

| | | Ag * | c_{Re} | c_{W} | c_{Cs} | c_{Li} | c_{S} | IMP_{K} ** | c_{K} *** |
|---|---|---|---|---|---|---|---|---|---|
| Examples | Support | [wt.-%] | [mmol/kg] | [mmol/kg] | [mmol/kg] | [mmol/kg] | [mmol/kg] | [mmol/kg] | [mmol/kg] |
| 1-1 ^{#} | A | 27.1 | 6.44 | 3.10 | 7.15 | 67.72 | 1.09 | 2.23 | 5.93 |
| 1-2 | A | 27.1 | 7.52 | 3.64 | 8.65 | 67.72 | 1.09 | 2.23 | 5.93 |
| 1-3 ^{#} | B | 27.1 | 6.44 | 3.10 | 7.15 | 67.72 | 1.09 | 2.23 | 5.57 |
| 1-4 | B | 27.1 | 7.52 | 3.64 | 8.65 | 67.72 | 1.09 | 2.23 | 5.57 |
| 1-5 ^{#} | B | 27.1 | 7.52 | 3.64 | 7.15 | 67.72 | 1.09 | 2.23 | 5.57 |
| 1-6 ^{#} | B | 27.1 | 5.00 | 3.00 | 6.50 | 67.72 | 1.09 | 2.23 | 5.57 |
| 1-7 ^{#} | B | 27.1 | 5.00 | 3.00 | 8.30 | 67.72 | 1.09 | 2.23 | 5.57 |
| 1-8 | B | 27.1 | 6.68 | 3.26 | 8.30 | 67.72 | 1.09 | 2.23 | 5.57 |
| 1-9 ^{#} | B | 27.1 | 7.20 | 3.60 | 6.50 | 67.72 | 1.09 | 2.23 | 5.57 |
| 1-10 | B | 27.1 | 7.20 | 3.60 | 8.30 | 67.72 | 1.09 | 2.23 | 5.57 |
| 1-11 ^{#} | B | 27.1 | 6.00 | 3.60 | 8.30 | 67.72 | 1.09 | 2.23 | 5.57 |

* Ag and all promoter values are calculated values
** IMP_{K} is understood to mean the amount of potassium added during impregnation and does not include the amount of potassium comprised in the alumina support prior to impregnation
*** c_{K} is understood to mean the total amount of potassium in the catalyst
^{#} comparative example

TABLE 2
Composition of Intermediate Products (Ag-contents are
reported in percent by weight of total catalyst, dopant
values are reported in mmol/kg of total catalyst).

| Intermediate | | Ag * | IMP_{K} ** | S_{K} **** | c_{K} *** |
|---|---|---|---|---|---|
| Product | Support | [wt.-%] | [mmol/kg] | [mmol/kg] | [mmol/kg] |
| IA | A | 18.0 | 1.41 | 4.19 | 5.61 |
| IB | B | 18.0 | 1.41 | 3.77 | 5.19 |

* Ag and all promoter values are calculated values
** IMP_{K} is understood to mean the amount of potassium added during impregnation and does not include the amount of potassium comprised in the alumina support prior to impregnation
* S_{K} is understood to mean the amount of potassium contributed to the intermediate product by the alumina support
**** c_{K} is understood to mean the total amount of potassium in the catalyst

TABLE 3
Amounts of ingredients used for preparation of catalysts 1-1 and 1-2.

| Catalyst | 1-1 ^{#} | 1-2 | 1-3 ^{#} | 1-4 | 1-5 ^{#} | 1-6 ^{#} |
|---|---|---|---|---|---|---|
| Ag-containing Intermediate | IA | IA | IB | IB | IB | IB |
| Amount of Ag-containing | 170.2 | 170.2 | 170.4 | 170.5 | 170.3 | 170.8 |
| Intermediate [g] | | | | | | |
| Amount of Ag-complex | 73.532 | 73.574 | 73.619 | 73.703 | 73.600 | 73.760 |
| solution [g] | | | | | | |
| Amount of promoter | 3.172 | 3.174 | 3.176 | 3.180 | 3.175 | 3.182 |
| solution I [g] | | | | | | |
| Li-/S-content in promoter | 2.85/0.212 | 2.85/0.212 | 2.85/0.212 | 2.85/0.212 | 2.85/0.212 | 2.85/0.212 |
| solution I [wt.-%] | | | | | | |
| Amount of promoter | 3.655 | 4.299 | 3.659 | 4.306 | 4.300 | 3.551 |
| solution II [g] | | | | | | |
| Cs-/W-content in promoter | 5.0/3.0 | 5.149/3.0 | 5.0/3.0 | 5.149/3.0 | 4.254/3.0 | 4.696/3.0 |
| solution II [wt.-%] | | | | | | |
| Amount of promoter | 6.239 | 7.283 | 2.311 | 2.699 | 2.696 | 1.797 |
| solution III [g] | | | | | | |
| Re-content in promoter | 3.7 | 3.7 | 10.0 | 10.0 | 10.0 | 10.0 |
| solution III [wt.-%] | | | | | | |
| Amount of DI H₂O [g] | 1.785 | 0.0 | 5.72 | 4.66 | 4.67 | 6.39 |

| Catalyst | 1-7 ^{#} | 1-8 | 1-9 ^{#} | 1-10 | 1-11 ^{#} |
|---|---|---|---|---|---|
| Ag-containing Intermediate | IB | IB | IB | IB | IB |
| Amount of Ag-containing | 170.7 | 170.3 | 170.5 | 170.5 | 170.3 |
| Intermediate [g] | | | | | |
| Amount of Ag-complex | 73.737 | 73.594 | 73.674 | 73.694 | 73.589 |
| solution [g] | | | | | |
| Amount of promoter | 3.181 | 3.175 | 3.179 | 3.180 | 3.175 |
| solution I [g] | | | | | |
| Li-/S-content in promoter | 2.85/0.212 | 2.85/0.212 | 2.85/0.212 | 2.85/0.212 | 2.85/0.212 |
| solution I [wt.-%] | | | | | |
| Amount of promoter | 3.549 | 3.851 | 4.253 | 4.254 | 4.248 |
| solution II [g] | | | | | |
| Cs-/W-content in promoter | 5.995/3.0 | 5.515/3.0 | 3.915/3.0 | 4.998/3.0 | 4.998/3.0 |
| solution II [wt.-%] | | | | | |
| Amount of promoter | 1.796 | 2.393 | 2.585 | 2.585 | 2.150 |
| solution III [g] | | | | | |
| Re-content in promoter | 10.0 | 10.0 | 10.0 | 10.0 | 10.0 |
| solution III [wt.-%] | | | | | |
| Amount of DI H₂O [g] | 6.38 | 5.43 | 4.85 | 4.84 | 5.28 |

^{#} comparative example

### Example 2—Catalyst Testing

An epoxidation reaction was conducted in a vertically-placed test reactor constructed from stainless steel with an inner diameter of 6 mm and a length of 2.2 m. The reactor was heated using hot oil contained in a heating mantle at a specified temperature. All temperatures below refer to the temperature of the hot oil. The reactor was filled with 9 g of inert steatite balls (0.8 to 1.1 mm), onto which 26.4 g of crushed catalyst screened to a desired particle size of 1.0 to 1.6 mm were packed, and thereon an additional 29 g of inert steatite balls (0.8-1.1 mm) were packed. The inlet gas was introduced to the top of the reactor in a “once-through” operation mode.

The catalysts were charged into the reactor at a reactor temperature of 90° C. under nitrogen flow of 130 NL/h at a pressure of 1.5 bar absolute. Then, the reactor temperature was ramped up to 210° C. at a heating rate of 50 K/h and the catalysts were maintained under these conditions for 15 h. Subsequently, the nitrogen flow was substituted by a flow of 114 NL/h methane and 1.5 NL/h CO₂. The reactor was pressurized to 16 bar absolute. Subsequently, 30.4 NL/h ethylene and 0.8 NL/h of a mixture of 500 ppm ethylene chloride in methane were added. Then, oxygen was introduced stepwise to reach a final flow of 6.1 NL/h. At this point, the inlet composition consisted of 20 vol.-% ethylene, 4 vol.-% oxygen, 1 vol.-% carbon dioxide, and ethylene chloride (EC) moderation of 2.5 parts per million by volume (ppmv), with methane used as a balance at the total gas flow rate of 152.7 NL/h.

The reactor temperature was ramped up to 225° C. at a heating rate of 5 K/h, and afterwards to 240° C. at a heating rate of 2.5 K/h. The catalysts were maintained under these conditions for 135 hours. Afterwards, EC concentration was decreased to 2.2 ppmv, and the temperature was decreased to 225° C. Subsequently, the inlet gas composition was gradually changed to 35 vol.-% ethylene, 7 vol.-% oxygen, 1 vol.-% carbon dioxide with methane used as a balance and a total gas flow rate of 147.9 NL/h. The temperature was adjusted to achieve an ethylene oxide (EO) concentration in the outlet gas of 3.05%. The EC concentration was adjusted to optimize the selectivity. Results of the catalyst tests are summarized in Table 4.

TABLE 4
Summary of catalyst tests.

| | | time on stream ^{§} | | | |
|---|---|---|---|---|---|
| | | 11 days | | 28 days | |
| | | | Heating | | Heating |
| | | EO- | Oil | EO- | Oil |
| | | Selec- | Temper- | Selec- | Temper- |
| | | tivity | ature | tivity | ature |
| Test | Catalyst | [%] | [° C.] | [%] | [° C.] |
| 2-1 | 1-1 ^{#} | n. d. * | n. d. * | 88.8 | 237.0 |
| 2-2 | 1-2 | n. d. * | n. d. * | 89.2 | 236.0 |
| 2-3 | 1-3 ^{#} | 86.8 | 230 | 87.7 | 232 |
| 2-4 | 1-4 | 87.1 | 232 | 88.0 | 234 |
| 2-5 | 1-5 ^{#} | 86.1 | 234 | 87.1 | 234 |
| 2-6 | 1-6 ^{#} | 86.9 | 231 | 87.7 | 231 |
| 2-7 | 1-7 ^{#} | 83.3 | 228 | 85.2 | 234 |
| 2-8 | 1-8 | 87.8 | 234 | 88.3 | 234 |
| 2-9 | 1-9 ^{#} | 85.3 | 227 | 85.9 | 228 |
| 2-10 | 1-10 | 87.6 | 235 | 88.4 | 236 |
| 2-11 | 1-11 ^{#} | 85.4 | 229 | n. d. * | n. d. * |

^{#} comparative example
* n. d. = not determined
^{§} time on stream: determined from the point of time of oxygen introduction

It is evident that inventive catalysts exhibit a higher ethylene oxide selectivity than comparative catalysts based on the same support.

### Example 3

Further catalyst compositions can be prepared by varying the amounts of silver, rhenium, tungsten, lithium or sulfur within the ranges disclosed above. The properties of catalyst compositions 2-1 to 2-8, all of which are based on support A and which are illustrated in Table 5 below, are expected to be substantially equally beneficial as those of the inventive catalyst compositions of example 2.

TABLE 5
Further catalyst compositions (Ag-contents are reported in percent by weight
of total catalyst, dopant values are reported in mmol/kg of total catalyst).

| | Ag | c_{Re} | c_{W} | c_{Cs} | c_{Li} | c_{S} | IMP_{K} * | c_{K} ** |
|---|---|---|---|---|---|---|---|---|
| Examples | [wt.-%] | [mmol/kg] | [mmol/kg] | [mmol/kg] | [mmol/kg] | [mmol/kg] | [mmol/kg] | [mmol/kg] |
| 2-1 | 29.5 | 7.09 | 3.35 | 7.71 | 68.00 | 1.00 | 2.28 | 5.86 |
| 2-2 | 29.5 | 7.09 | 3.35 | 7.71 | 68.00 | 1.40 | 2.28 | 5.86 |
| 2-3 | 29.5 | 7.09 | 3.35 | 7.71 | 68.00 | 0.60 | 2.28 | 5.86 |
| 2-4 | 29.5 | 7.09 | 3.35 | 7.71 | 75.00 | 1.00 | 2.28 | 5.86 |
| 2-5 | 29.5 | 7.09 | 3.35 | 7.71 | 61.00 | 1.00 | 2.28 | 5.86 |
| 2-6 | 29.5 | 7.09 | 3.35 | 7.71 | 68.00 | 1.00 | 3.32 | 6.90 |
| 2-7 | 29.5 | 7.09 | 3.35 | 7.71 | 68.00 | 1.00 | 1.32 | 4.90 |
| 2-8 | 29.5 | 7.52 | 3.86 | 8.65 | 68.00 | 1.00 | 2.28 | 5.86 |

* IMP_{K} is understood to mean the amount of potassium added during impregnation and does not include the amount of potassium comprised in the alumina support prior to impregnation
** c_{K} is understood to mean the total amount of potassium in the catalyst

1.-13. (canceled)

14. An epoxidation catalyst comprising silver, cesium, rhenium, and tungsten deposited on an alumina support,
wherein the catalyst comprises 20 to 50 wt.-% of silver, relative to the weight of the catalyst, an amount of cesium c_{Cs} of at least 7.5 mmol per kg of catalyst, and an amount of rhenium c_{Re} and an amount of tungsten c_{W} so as to meet the following requirements:
c_{Re}≥6.7 mmol per kg of catalyst; and
c_{Re}+(2×c_{W})≥13.2 mmol per kg of catalyst.

15. The catalyst according to claim 14, comprising an amount of tungsten c_{W} of at least 3.2 mmol per kg of catalyst.

16. The catalyst according to claim 14, comprising an amount of rhenium c_{Re} of 6.7 to 10.0 mmol per kg of catalyst.

17. The catalyst according to claim 14, comprising an amount of tungsten c_{W} of 3.2 to 5.4 mmol per kg of catalyst.

18. The catalyst according to claim 14, comprising an amount of cesium c_{Cs} of 7.5 to 12.4 mmol per kg of catalyst

19. The catalyst according to claim 14, comprising an amount of lithium c_{Li} of at least 14.0 mmol per kg of catalyst.

20. The catalyst according to claim 14, comprising an amount of sulfur c_{S} of 10.0 mmol or less per kg of catalyst.

21. The catalyst according to claim 14, comprising an amount of potassium c_{K} of 12.0 mmol or less per kg of catalyst.

22. The catalyst according to claim 14, wherein the alumina support comprises at least 80 wt.-% alpha-alumina.

23. The catalyst according to claim 14, wherein the catalyst has a BET surface area in the range of 1.6 to 5.0 m²/g.

24. The catalyst according to claim 14, wherein the catalyst has a total Hg pore volume of 0.15 to 1.0 mL/g, as determined by mercury porosimetry.

25. A process for preparing an epoxidation catalyst as defined in claim 14, comprising
i) impregnating an alumina support with a silver impregnation solution; and
ii) subjecting the impregnated refractory support to a calcination process;
wherein steps i) and ii) are optionally repeated, and at least one silver impregnation solution comprises rhenium, tungsten and cesium.

26. A process for producing ethylene oxide by gas-phase oxidation of ethylene, comprising reacting ethylene and oxygen in the presence of an epoxidation catalyst according to claim 14.
